(19) United States
(12) Patent Application Publication
(10) Pub. No.: US 2001/0025157 A1
(43) Pub. Date: Sep. 27, 2001

(54) Implantable dispensing device for controllably dispensing medicinal fluid

(76) Inventor: Marshall S. Kriesell, St. Paul, MN (US)

Correspondence Address: James E. Brunton, Suite 860, 700 North Brand Blvd., P.O. Box 29000, Glendale, CA, 91029, US

(21) Appl. No.: 09/760,128

(22) Filed: Jan. 12, 2001

Related U.S. Application Data
(63) Continuation-in-part of application No. 09/387,447, filed on Sep. 1, 1999, now Pat. No. 6,174,300.
Application No. 09/387,447 is a division of application No. 08/919,147, filed on Aug. 27, 1997, now Pat. No. 5,961,492.

(51) Int. Cl. A61M 37/00; A61K 9/22

(52) U.S. Cl. 604/132; 604/892.1

(57) ABSTRACT

A compact, small-volume, very low flow rate fluid dispensing device for precisely dispensing medicinal fluids into a patient at a very low, controlled flow rate over long periods of time. The dispensing device is readily implantable into the patient's body and includes a novel heat stimulatable polymer gel material which uniquely functions as an internal energy source for controllably expelling the medicinal fluids from the device.

### BACKGROUND OF THE INVENTION

This is a continuation-in-part of co-pending Divisional application Ser. No. 09/387,447 filed Sep. 1, 1999; which is a Divisional application of copending application Ser. No. 08/919,147 filed Aug. 27, 1997, now U. S. Pat. No. 5,961,492.

### FIELD OF THE INVENTION

The present invention relates generally to fluid delivery devices. More particularly, the invention concerns an improved apparatus for delivery of medicinal fluid to a patient that includes an internally disposed energy source that can be stimulated by the heat of the patient's body to controllably expel the fluid from the apparatus.

### DISCUSSION OF THE INVENTION

The oral route is the most frequent route of drug administration. Oral administration is relatively easy for most patients and rarely causes physical discomfort. However, many medicinal agents require a parenteral route of administration thus bypassing the digestive system and precluding degradation by the catalytic enzymes in the digestive tract and the liver. The use of more potent medications at elevated concentrations has also increased the need for accuracy in controlling the delivery of such drugs. The delivery device, while not an active pharmacologic agent, may enhance the activity of the drug by medicating its therapeutic effectiveness. Certain classes of new pharmacologic agents possess a very narrow range of therapeutic effectiveness, for instance, too small a dose results in no effect, while too great a dose results in toxic reaction.

In the past, prolonged infusion of fluids has generally been accomplished using gravity flow means coupled with electronic based controls and typically involved the use of intravenous administration sets and the familiar bottle or solution bag suspended above the patient. Such methods are cumbersome, imprecise and, generally non-ambulatory requiring bed confinement of the patient. Periodic monitoring of the apparatus by the nurse or doctor is required to detect malfunctions of the infusion apparatus.

A family of highly unique fluid delivery devices has been developed by the present inventor. These novel devices make use of various types of energy sources that are housed within a generally cylindrically shaped housing and function to controllably expel various types of medicaments to the patient. U.S. Pat. No. 5,743,879 issued to the present inventor, describes one such device. A similar device is disclosed in U.S. Pat. No. 6,030,363 also issued to the present inventor. U.S. Pat. No. 5,205,820 issued to the present inventor discusses adding means of the character discussed in one form of the present invention and should be consulted in this regard. U.S. Pat. No. 5,961,492 entitled Fluid Delivery Device with Temperature Controlled Energy Source, in which the present inventor is named as a co-inventor is also pertinent to a complete understanding of the present invention. Accordingly, U.S. Pat. No. 5,961,492 is also incorporated by reference as though fully set forth herein. An application filed by the present inventor on Sep. 1, 1999 namely U.S. Ser. No. 09/387,447, is also pertinent to fully understanding the present invention. Accordingly, application Ser. No. 09/387,447 is incorporated by reference as though fully set forth herein.

The apparatus of the present invention, comprises a very small, generally cylindrically shaped implantable unit that makes use of novel heat stimulatable polymer gel materials as energy sources for delivery to the patient of medicinal fluids. By way of example, the apparatus of the invention can be used for the controlled delivery of a variety of beneficial agents such as biopharmaceuticals and like agents.

### SUMMARY OF THE INVENTION

It is an object of the present invention to provide a compact, small volume, low flow rate fluid dispensing device for delivering medicinal fluids such as specialized oncolytic agents and other injectable biopharmaceuticals to a specific delivery site. The dispensing device is readily implantable into the patient's body and includes novel heat stimulatable polymer gel materials which uniquely function as internal energy sources for controllably expelling the medicinal fluids from the device.

Another object of the invention is to provide an implantable fluid dispensing device that can be used for the precise delivery of various beneficial agents, including mixtures of medicinal fluids and immobilized drugs, into the patient at ultra low controlled flow rates over very long periods of time.

Another object of the invention is to provide an apparatus as described in the preceding paragraph that includes adding means for adding various compounds such as drugs to a fluid contained within the fluid reservoir of the device. In this connection, the various compounds are uniquely removably connected to presentation means, such as scaffolds that are carried within the device housing.

Another object of the invention is to provide an apparatus of the aforementioned character that is of a simple generally cylindrically shaped construction that is highly reliable in operation for the small volume, ultra low flow rate delivery of beneficial agents to a patient.

Another object of the invention is to provide an apparatus that embodies as its stored energy source, a soft, pliable, semi-solid, thermo-responsive mass that is controllably heated by the heat of the patient's body in a manner to expel a low volume dose of medication to the patient at precisely controlled rates.

Another object of the invention is to provide an apparatus as described in the preceding paragraph in which the heat expandable mass is specifically tailored to provide precise, predictable protocol delivery of the medicinal agent stored within the reservoir of the device.

Another object of the invention is to provide an apparatus of the character described in which the thermo-responsive stored energy source is constructed from various types of polymeric materials such as phase transition gels.

Another object of the invention is to provide an implantable device of the character described that includes flow rate control means for precisely controlling the rate of fluid flow toward the patient.

Another object of the invention is to provide a device as previously described, which includes novel sealing means for sealing the fluid reservoir until time of us.

Another object of the invention is to provide an implantable fluid delivery device as described in the preceding paragraphs that includes novel infusion means for delivering medicinal agents to a therapeutic site remote from the location of implant within the patient's body.

### BRIEF DESCRIPTION OF THE DRAWINGS

FIG. 1 is a generally perspective view of one form of implantable medicament dispensing device of the invention.

FIG. 2 is a generally perspective, exploded view of the device shown in FIG. 1.

FIG. 3 is an enlarged, side-elevational, cross-sectional view of the device of FIG. 1.

FIG. 3A is a fragmentary, cross-sectional view of an alternate form of the device shown in FIG. 3.

FIG. 4 is a cross-sectional view taken along lines 4-4 of FIG. 3.

FIG. 4A is a cross-sectional view taken along lines 4A-4A of FIG. 3A.

FIG. 5 is a cross-sectional view taken along lines 5-5 of FIG. 3.

FIG. 6 is a generally perspective view of an alternate form of the dispensing device of the invention.

FIG. 7 is a generally perspective, exploded view of the device shown in FIG. 6.

FIG. 8 is a side-elevational, cross-sectional view of the device shown in FIG. 6.

FIG. 9 is a cross-sectional view taken along lines 9-9 of FIG. 8.

FIG. 10 is a fragmentary, cross-sectional view of the right hand portion of the device as viewed in FIG. 8.

FIG. 11 is a generally perspective illustrative view showing the device implanted within the patient's body.

FIG. 12 is a generally perspective view of an alternate form of the dispensing device of the invention.

FIG. 13 is a generally perspective, exploded view of the device shown in FIG. 12.

FIG. 14 is a side-elevational, cross-sectional view of the device shown in FIG. 12.

FIG. 15 is a cross-sectional view taken along lines 15-15 of FIG. 14.

FIG. 16 is a cross-sectional view taken along lines 16-16 of FIG. 14.

FIG. 17 is a generally perspective view of still another alternate form of the dispensing device of the invention.

FIG. 18 is a generally perspective, exploded view of the device shown in FIG. 17.

FIG. 19 is a front view of the device shown in FIG. 17.

FIG. 20 is a side-elevational, cross-sectional view of the device taken along lines 20-20 of FIG. 19.

FIG. 21 is a cross-sectional view taken along lines 21-21 of FIG. 20.

FIG. 22 is a cross-sectional view taken along lines 22-22 of FIG. 20.

FIG. 23 is a cross-sectional view taken along lines 23-23 of FIG. 20.

FIG. 24 is a cross-sectional view taken along lines 24-24 of FIG. 20.

FIG. 25A is a generally perspective illustrative view of one form of additive presentation means or scaffold of the invention.

FIG. 25B is a generally perspective illustrative view of another form of additive presentation means or scaffold of the invention.

FIG. 25C is a generally perspective illustrative view of still another form of additive presentation means or scaffold of the invention.

FIG. 25D is a generally perspective illustrative view of yet another form of additive presentation means or scaffold of the invention.

FIG. 25E is a generally perspective illustrative view of another form of additive presentation means or scaffold of the invention.

FIG. 25F is a generally perspective illustrative view of another form of additive presentation means or scaffold of the invention.

FIG. 25G is a generally perspective illustrative view of another form of additive presentation means or scaffold of the invention.

FIG. 25H is a generally perspective illustrative view of yet another form of additive presentation means or scaffold of the invention.

FIG. 25I is a generally perspective illustrative view of another form of additive presentation means or scaffold of the invention.

FIG. 25J is a generally perspective illustrative view of another form of additive presentation means or scaffold of the invention.

FIG. 25K is a generally perspective illustrative view of another form of additive presentation means or scaffold of the invention.

FIG. 25L is a generally perspective illustrative view of another form of additive presentation means or scaffold of the invention.

FIG. 25M is a generally perspective illustrative view of another form of additive presentation means or scaffold of the invention.

FIGS. 26A, 26B, 26C, and 26D are general diagrammatic views illustrating various means for affinity attachment of ligands, protein molecules and enzymes to selected substrates.

### DESCRIPTION OF THE INVENTION

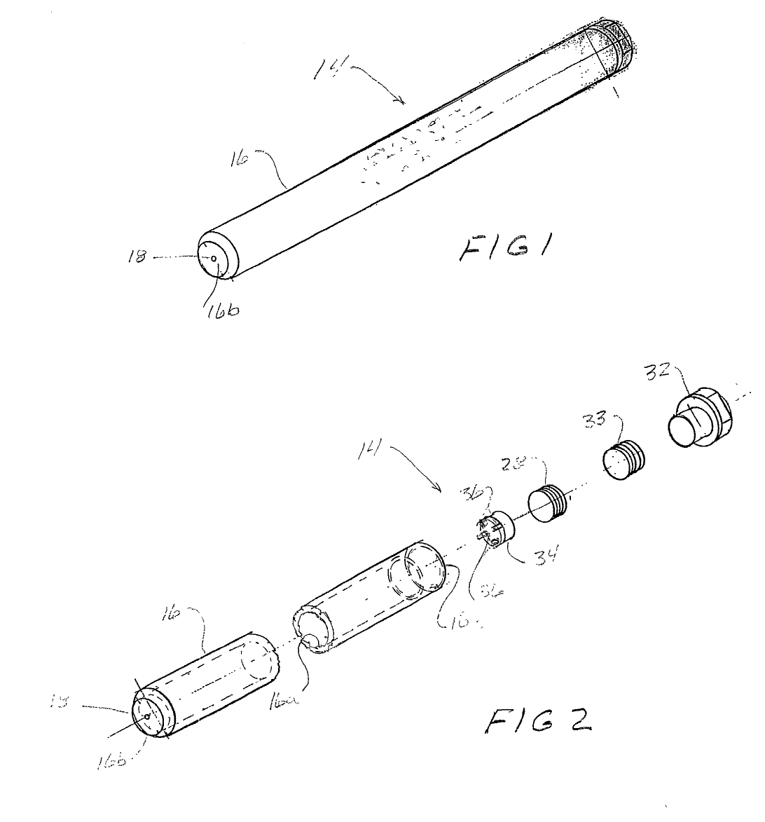

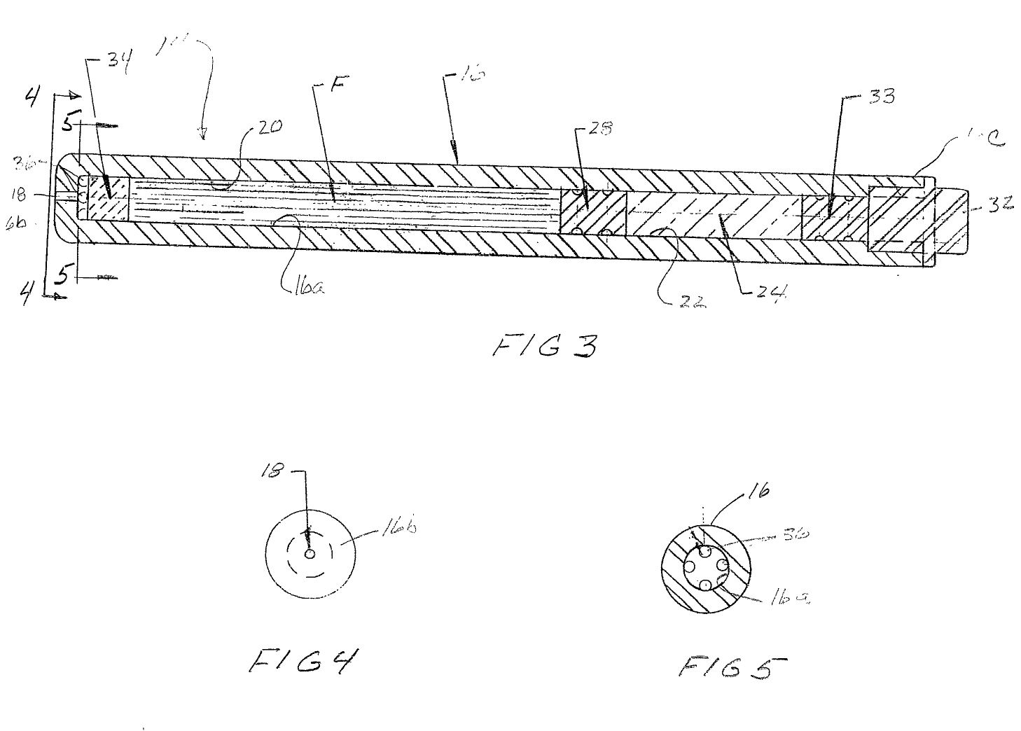

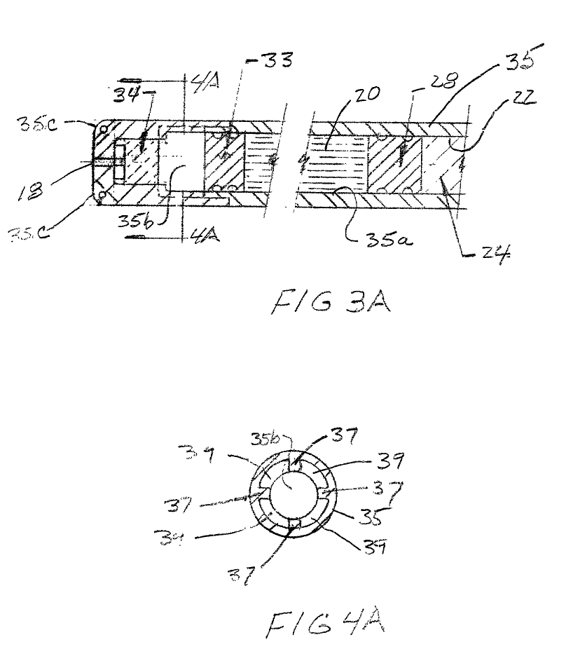

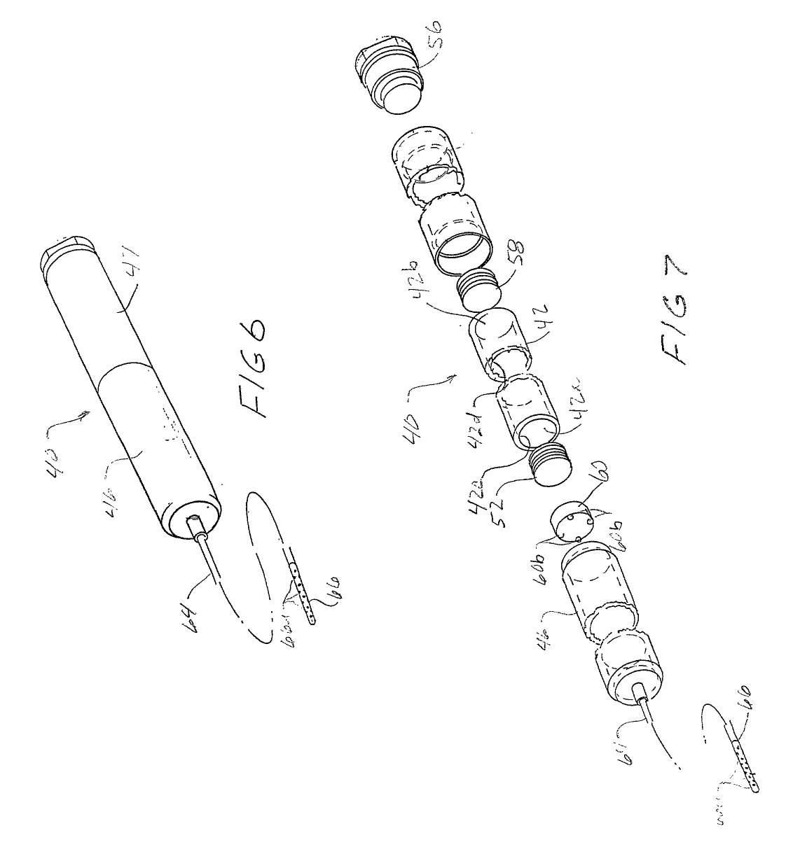

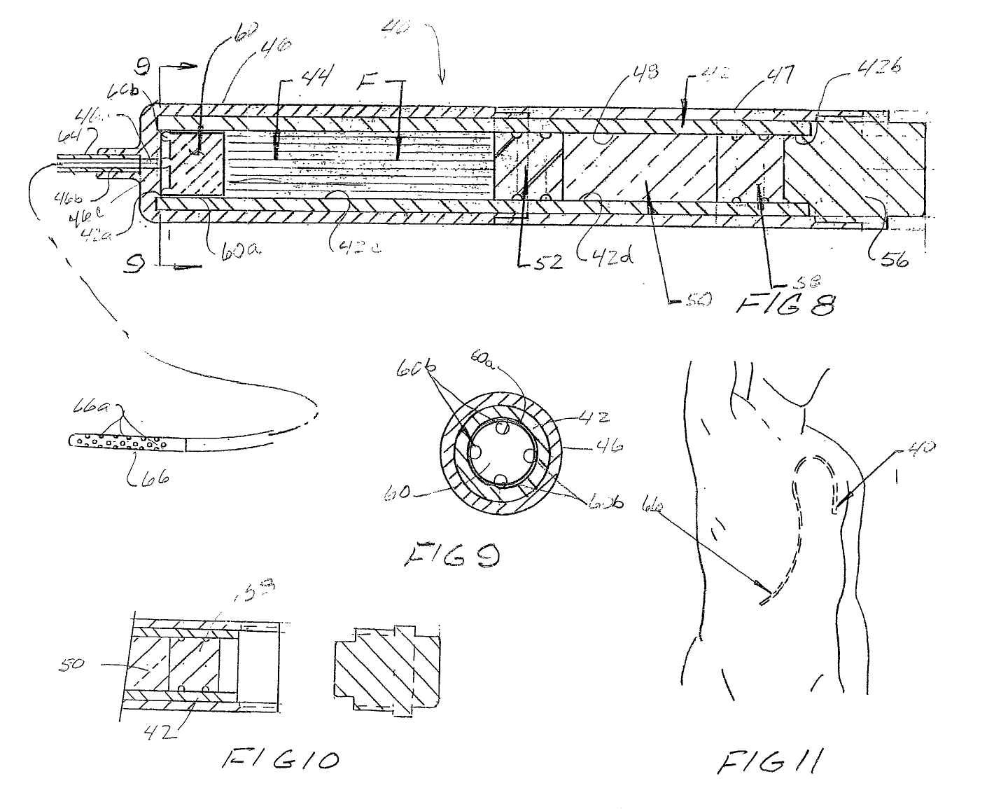

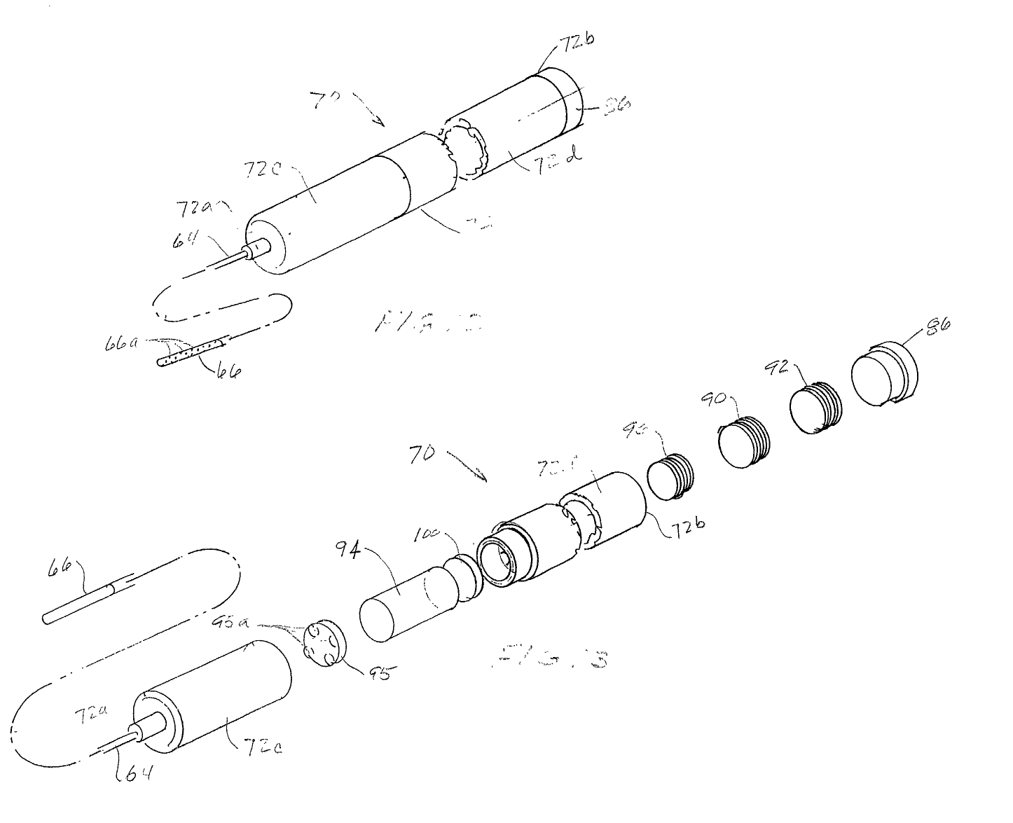

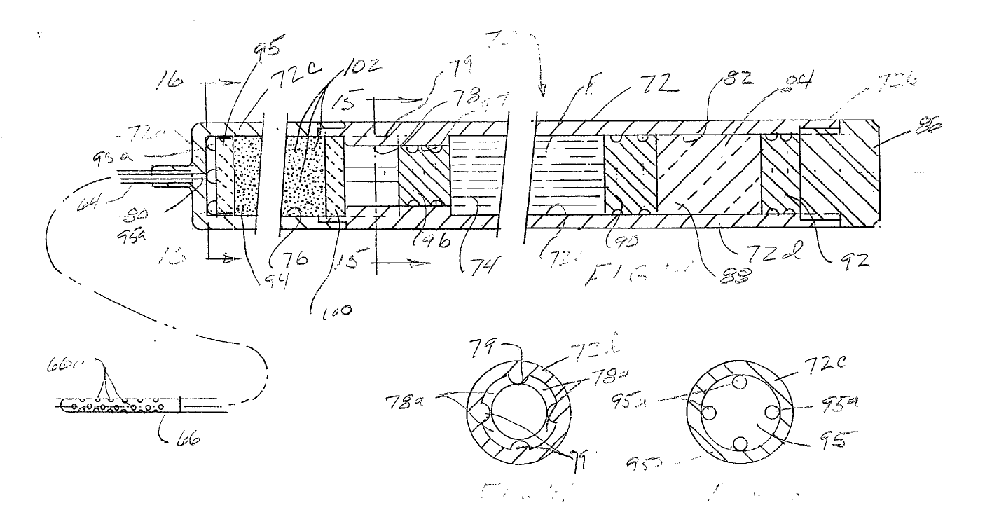

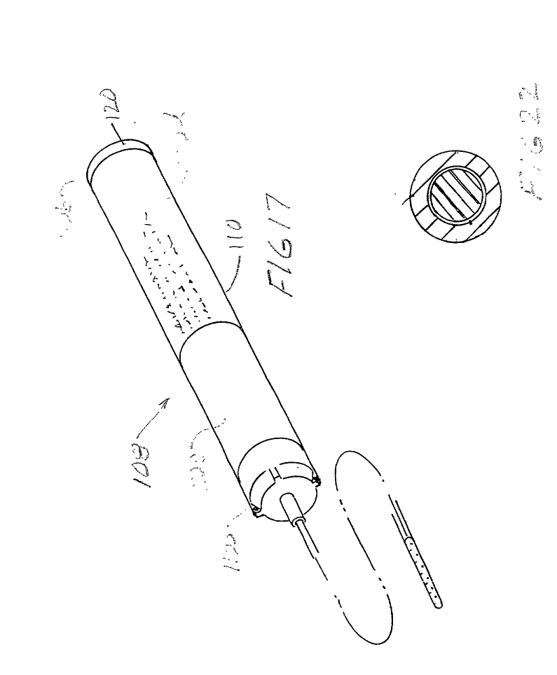

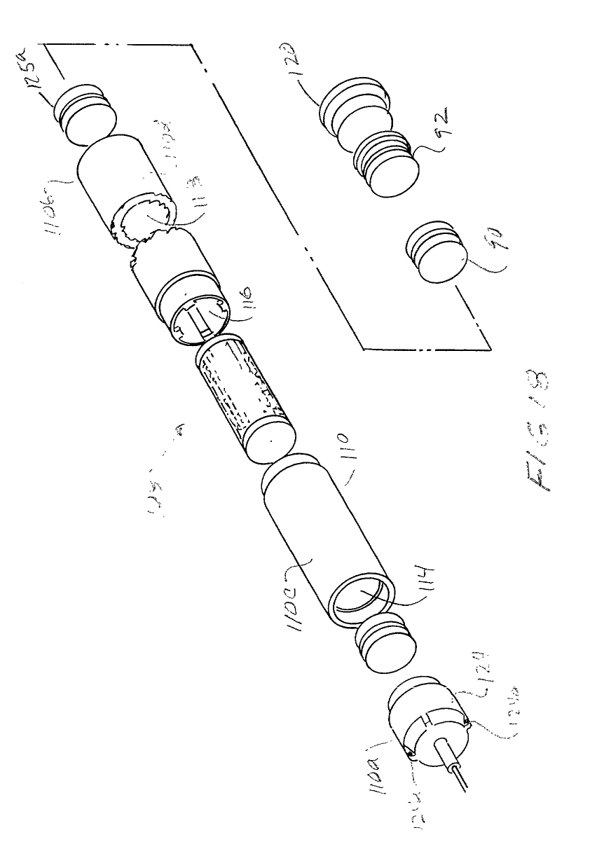

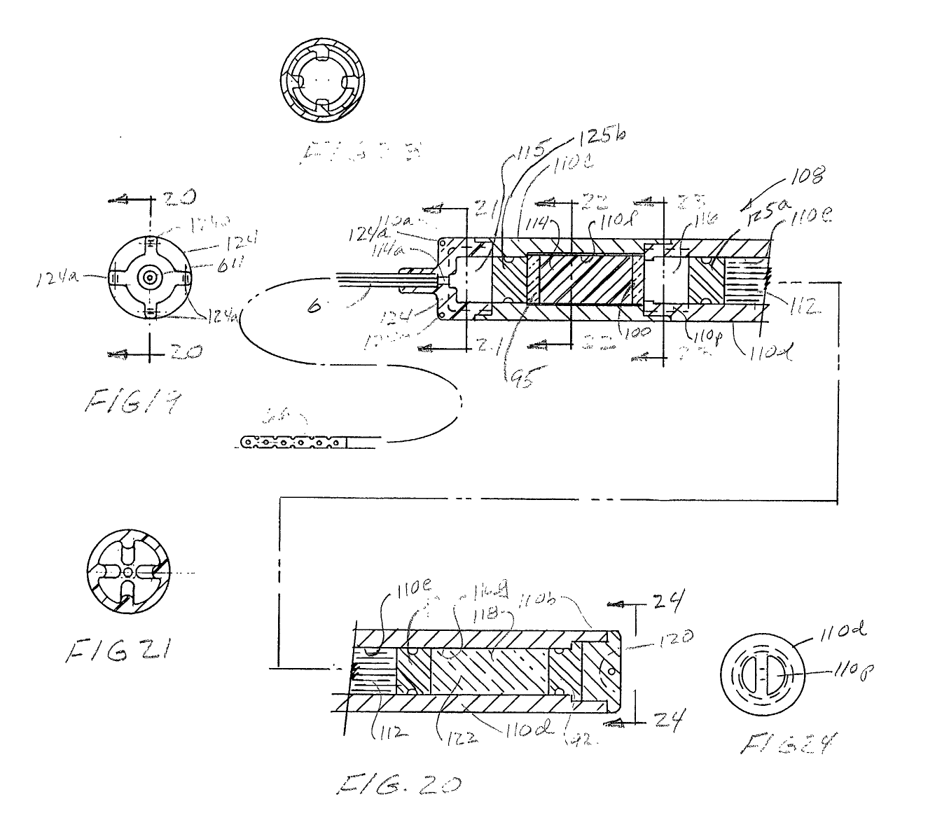

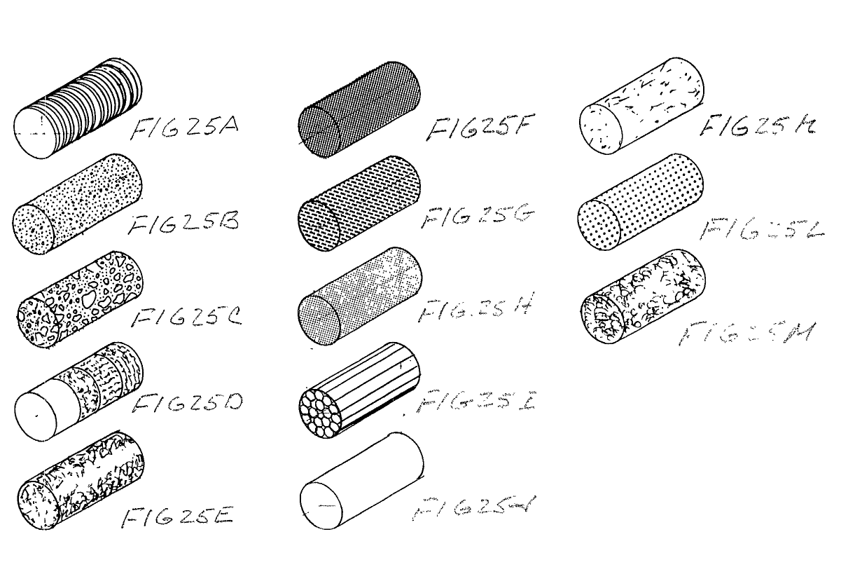

Referring to the drawings and particularly to FIGS. 1 through 5, one form of the implantable fluid delivery device of the invention for delivering medicinal fluids to a patient is there shown and generally designated by the numeral 14. In this form of the invention, the delivery device comprises an elongated housing 15 that includes a tubular member 16. Housing has a fluid outlet 18 and an interior wall 16a formed by tubular member 16 that defines a first reservoir 20 that is in communication with fluid outlet 18 in the manner best seen in FIG. 3. Interior wall 16a of tubular member 16 also defines a second reservoir 22.

First reservoir 20 functions to contain the beneficial agent to be delivered to the patient as, for example, the medicinal fluid “F” or other flowable substance. Reservoir 22, on the other hand, contains a novel heat expandable stored energy means, shown here as an expandable mass 24, that is slowly expandable over time by the heat of the patient's body.

Disposed between reservoir or chamber 20 and reservoir or chamber 22 is a pusher means for urging the medicinal fluid contained within reservoir 20 toward the fluid outlet 18 upon expansion of expandable mass 24. In the present form of the invention, this pusher means comprises an elastomeric pusher member 28 that is disposed in sealing engagement with the internal wall of tubular member 16.

Tubular member 16 includes a first closed end 16b and a second open end 16c. As best seen in FIG. 3, the fluid outlet is formed in first end 16b of the tubular member and here comprises a small diameter, generally cylindrically shaped orifice 18. Open end 16c of tubular member 16 is closed by a closure cap 32, a portion of which is hermetically, sealably received within open end 16c. Located between closure cap 32 and expandable mass 24 is an elastomeric stopper member 33 that is disposed in sealable engagement with the internal wall of tubular member 16.

Forming an important aspect of the delivery device of the present invention is flow control means for controlling the rate of fluid flow of medicinal fluid from reservoir 20 through fluid outlet 18. In the present form of the invention, this flow control means comprises a porous rate control frit 34 that is sealably positioned proximate reservoir 20 and in a spaced-apart location from the inner wall of first end 16b of tubular member 16. To maintain the frit in a spaced-apart position, a plurality of circumferentially spaced, standoff elements 36 are provided on frit 34 (FIG. 5).

With regard to the heat expandable mass 24, this novel stored energy source can take several forms, but a particularly attractive form for devices of the present invention is a semisolid form such as a gel. Unlike a liquid, which can offer no perm anent resistance to change in shape and must be constrained within some type of container, expandable mass 24 is of a semisolid form which can advantageously be handled without external containment under ambient manufacturing conditions. From a technical viewpoint, gels are often characterized as semi-solids, which reside in a state between a liquid and a solid state. Frequently gels comprise a crosslinked network of long polymer molecules with liquid molecules trapped within the network. Many gels known in the prior art not only are capable of significantly large volume change in response to stimulus (phase-transition gels), but also exhibit physical characteristics that enable them to closely conform to the shape of an adjacent member.

Phase transition gels best suited for use in constructing the heat expandable means of the present invention are gels which exhibit a large volume change at a given phase-transition condition. Unlike liquids, which exhibit a fixed temperature for state of vaporization to a known volume and with such vaporization point changing as a function of ambient pressure, the phase-transition gels in this invention are multicomponent polymers which can be made to respond with various volume changes to a singular external temperature stimuli.

Advantageously, the difference in volume between the expanded phase of these phase-transition gels and the contracted phase thereof can be orders of magnitude. Examples of suitable phase-transition gels are disclosed in Tanaka et al., U.S. Pat. No. 4,732,930; No. Re-35068 and No. 5,403,893. Because of the pertinence of these patents, U.S. Pat. No. 4,732,930, U.S. Pat. No. 5,403,893 and U.S. Pat. No. Re-35068 are all hereby incorporated by reference as though fully set forth herein.

While a number of the phase-transition gels described in the Tanaka et al patents can be used to construct the heat expandable stored energy means of the present invention, the ionized acrylamide gel compositions therein described are desirable in many applications because of the quite drastic volume change they exhibit in response to an external stimulus such as the body temperature of the patient. These ionized acrylamide gel compositions comprise a cross-linked, partially ionized polyacrylamide gel wherein between up to 20% of the amide groups are hydrolyzed to carboxyl groups. The gel includes a solvent of a critical concentration at which even a slight change in temperature, pH or salt concentration causes the gel to shrink or swell dramatically. As pointed out by Tanaka et al in the aforementioned patents, the particular critical concentration utilized in the gel composition depends upon the solvent employed, the temperature of the gel and the degree of hydrolysis of the gel. The gel also can contain a positive metal ion such as sodium or magnesium which has the effect of increasing the change in gel volume caused by change of solvent concentration, temperature, pH or, salt concentration.

Another form of phase-transition gel suitable for use in the apparatus of the present invention comprises interpenetrating polymer networks that include a first polymer and a second polymer wherein the second polymer interpenetrates the first polymer. Suitable first and second polymers include polymers that can interact during exposure to a phase-transition condition to thereby cause a significantly large volume change of the gel. Suitable interpenetrating polymer networks can also include more than two polymers. For example, additional polymers can be included in the network which interpenetrate the first and/or second polymers. The nature of these polymers as well as the nature of the interaction between the polymers is discussed in detail in Tanaka, U.S. Pat. No. 5,403,893, and will not here be repeated.

The responsive gels may also be reversibly responsive. For example, such gels experience certain environmental changes, the entire gel, or a component thereof will undergo a reversible volumetric change which typically involves a shift between two equilibrium states as, for example, expanded and collapsed. This reversible volume change of the entire gel, or a component of the gel may be either continuous or discontinuous. Typically, a continuous volume change is marked by a reversible change in volume that occurs over a substantial change in environmental condition. On the other hand, the gel, or a component thereof, may undergo a discontinuous volume change in which the reversible transition from expanded to collapsed states, and back again, typically occurs over a relatively small change in environmental condition. A gel undergoing a continuous phasetransition may have a similar order of magnitude total volume change as a gel undergoing a discontinuous phase-transition.

Typically, volumetric changes in the phase transition gels result from competition between intermolecular forces, usually electrostatic in nature. Such volumetric changes are believed to be driven primarily by four fundamental forces, that is ionic, hydrophobic, hydrogen bonding and van der Waals bonding interactions, either alone or in combination. Changes in temperature most strongly affect hydrophobic interactions and hydrogen bonding.

Of particular interest is the fact that gels consisting of copolymers of positively and negatively charged groups may be formulated so that the volume change is governed by more than one fundamental force. In these gels, polymer segments typically interact with each other through ionic interactions and hydrogen bonding.

By way of summary, gels suitable for use as the stored energy sources of the present invention include various cross-linked polymers and gels which can be synthesized from the polymerization of a monomer and a cross-linking agent.

More particularly, suitable gels can be made from any polymer with side groups that can react with a di-or multi-functional cross-linking molecule. However, the simplest system from which gels can be made are polymers with hydroxyl, acid or amine side groups.

By way of non-limiting example, suitable gels for use as the stored energy means may consist, in whole or in part, of polymers made by copolymerization/cross linking of monofunctional and polyfunctional polymerizable vinyl monomers. The monomer may include N, N-disubstituted acrylamides such as N,N-dialkylsubstituted acrylamides, or di-N,N substituted acrylamides where the disubstitution form part of a ring, acrylate ethers, alkyl substituted vinyl ethers, glycol ethers, and mixtures thereof.

Exemplary polymeric gel networks thus may contain poly (N,N-dialkylacrylamide), poly(ethyl acrylate) and mixtures thereof, as well as polymers of N-alkylacrylamide (or analogous N-alkylmethacrylamide) derivatives such as N-ethylacrylamide, N-n-propylacrylamide, N-n-propylmethylacrylamide, or various acrylate copolymers.

Exemplary cross-linking agents may include ethylene glycol diacrylate (EGDA); di(ethylene glycol)bis(allyl carbonate) (“DEGBAC”); methylenebis(acrylamide) (“bis”); ethylene glycol dimethacrylate (“EGDMA”); magnesium methacrylate (“MgMA₂”); and mixtures thereof. Cross-linkers suitable for polymeric precursors may include diglycidyl ether, divinyl sulfone, epichlorohydrin, phosphoryl chloride, trimetaphosphate, trimethylomelamine, polyacrolein, and ceric ion redox systems, although the most preferred of these will not have active hydrogens. The cross-linking agent effects partial cross-linking of the polymer and provides a means to control the gel's mechanical strength, swelling degree, and intensity of volume change trigger by changing the cross-linking density. Crosslinking of linear polymers by chemical reagents is preferred for gels made from biological polymers such as cellulose ethers. Preferred cross-linkers for polysaccharide gels, especially cellulose ethers, are multifunctional carboxylic acids, such as adipic acid (hexanedioic acid: HOOC(CH₂)₄COOH), succinic acid (HOOC(CH₂)₂COOH), malonic acid (propanedioic acid: CH₂(COOH)₂, sebacic acid (decanedioic acid: HOOC(CH₂)COOH), glutaric acid (pentanedioic acid: HOOC(CH₂)₃COOH), or 1, 10 decanedicarboxylic acid.

In using the fluid delivery device of the invention, the device is suitably implanted in the patient's body in the manner well understood by those skilled in the art with fluid outlet 18 strategically positioned at the location at which the medicinal fluid is to be delivered to the patient. Once implanted, the heat of the patient's body will cause expandable mass 24 to controllably expand causing plunger 28 to be urged forwardly of reservoir 20. As the plunger moves forwardly, the fluid “F” will flow through rate control frit 34 and will be forced out of outlet 18 where it will infuse the location selected by the treating physician. As previously mentioned, rate control frit 34 functions to provide a very low, precisely controlled rate of fluid flow toward outlet 18 over long periods of time.

Referring next to FIGS. 3A and 4A, an alternate form of the implantable fluid delivery device of the invention is there shown. This form of the invention is similar in many respects to that shown in FIGS. 1 through 5 and like numerals are used in FIGS. 3A and 4A to identify like components. As before, the delivery device comprises an elongated housing that includes a tubular member 35 having a fluid outlet 18 and an interior wall 35a that defines a first reservoir 20 that is in communication with fluid outlet 18 in the manner best seen in FIG. 3A. Interior wall 35a of tubular member 35 also defines a second reservoir 22.

As in the earlier described embodiment, first reservoir 20 functions to contain the beneficial agent to be delivered Reservoir 22, on the other hand, contains a novel heat expandable stored energy means, shown here as an expandable mass 24, that is identical to that previously described. The novel feature of this latest form of the invention resides in the provision of sealing means, the character of which will presently be described for sealing first reservoir 20 from atmosphere until time of use of the device.

Disposed between reservoir or chamber 20 and reservoir or chamber 22 is a pusher means of elastomeric pusher member 28 for urging the medicinal fluid contained within reservoir 20 toward the fluid outlet 18 upon expansion of expandable mass 24. However, in this latest form of the invention, a second pusher member 33 is provided and is disposed in a spaced-apart location from rate control frit 34 and in sealing engagement with the internal wall 35a of tubular member 35.

Second pusher member here comprises a part of the fluid reservoir sealing means of this latest form of the invention and functions to maintain the medicament reservoir 20 sealed until time of implantation and use. More particularly, as shown in FIGS. 3A and 4A tubular member 35 is provided proximate its forward end with a chamber 35b having circumferentially spaced standoffs 37 that define therebetween circumferentially spaced fluid flow channels 39. With this novel construction, once the device is implanted, the heat of the patient's body will cause expandable mass 24 to controllably expand causing plunger 28 to be urged forwardly of reservoir 20. As the plunger moves forwardly, it will move second plunger 33 into chamber 35b and into engagement with standoffs 37 allowing the fluid “F” to flow through flow channels 39 and then through rate control frit 34 where it will be forced out of outlet 18 that is disposed at a location selected by the treating physician. However, until the time of use, plunger 33 will uniquely serve to maintain medicament reservoir 35a substantially sealed from atmosphere.

It should also be observed that in this latest form of the invention, tubular member 35 is provided with apertured suture links 35c for tying the device in place during the implantation step.

Turning next to FIGS. 6 through 11, an alternate form of the implantable fluid delivery device of the invention for delivering medicinal fluids to a patient is there shown and generally designated by the numeral 40. This latest form of the invention comprises an elongated, tubular-shaped glass housing 42 having first and second open ends 42a and 42b respectively. Housing 42 has an interior wall 42c defining a first reservoir 44 that is in communication with fluid outlet 46a formed in a biocompatible front cover 46 (FIG. 8). Similarly, housing 42 has an interior wall 42d that defines a second reservoir 48. A biocompatible rear cover 47 covers the rear end portion of housing 42 in the manner shown in FIG. 8.

First reservoir 44 functions to contain the medicinal fluid “F” that is to be delivered to the patient. Reservoir 48, on the other hand, contains a heat expandable mass 50 that is of identical character to expandable mass 24 as previously described and is readily expandable by the heat of the patient's body.

Disposed between reservoir 44 and reservoir 48 is a pusher means for urging the medicinal fluid contained within chamber 44 toward the fluid outlet 46a upon expansion of expandable mass 50. In the present form of the invention, this pusher means comprises an elastomeric pusher member 52 that is disposed in slidable sealing engagement with the internal wall 42c of tubular member 42.

As best seen in FIG. 8, fluid outlet 46a here comprises a small diameter generally cylindrically shaped orifice that communicates with the infusion means of the device, the character of which will presently be described. Open end 42b of tubular member 42 is closed by a closure cap 56, a portion of which is hermetically, sealably received within open end 42b. Located between closure cap 56 and expandable mass 50 is an elastomeric stopper member 58 that is disposed in sealable engagement with the internal wall 42d of tubular member 42.

Forming an important aspect of the delivery device of this latest form of the invention is flow control means for controlling the rate of fluid flow of medicinal fluid from chamber 44 through fluid outlet 46a. This flow control means here comprises a porous rate control frit 60 that is sealably positioned proximate reservoir 44 and in a spaced-apart location from the inner wall of closed end 46c of front overcover 46. Frit 60 includes an elastomeric coating 60a that seals against the inner wall 42c. To maintain the frit in this spaced-apart location, a plurality of circumferentially spaced standoff elements 60b are provided on frit 60.

As illustrated in FIG. 6, the infusion means of the invention for infusing medicinal fluid into the patient comprises an elongated delivery catheter 64, the proximal end of which is connected to cover 46. Catheter 64 is received within a socket-like opening 46b formed in front cover 46 and communicates with outlet 46a in the manner depicted in FIG. 8. Connected to the distal end of delivery catheter 64 is a porous delivery tip 66 that has a multiplicity of fluid passageways 66a that permit the medicinal fluid to flow uniformly outwardly of the tip. It should be understood that for certain applications, delivery tip 66 could be provided with a single delivery orifice.

In using the fluid delivery device of the invention, the device is implanted in the patient's body in the manner shown in FIG. 11 with the porous tip 66 strategically positioned at the location at which the beneficial agent is to be delivered to the patient. Once implanted, the heat of the patient's body will cause expandable mass 50 to controllably expand causing plunger 52 to be urged forwardly of reservoir 44. As the plunger moves forwardly, the fluid “F” will flow through rate control frit 60 and will be forced out of outlet 46a and into delivery catheter 64. The fluid will then flow through the delivery catheter and outwardly of porous tip 66 at a precisely controlled low delivery rate where it will infuse the location selected by the treating physician.

Referring to FIGS. 12 through 16, another form of the implantable fluid delivery device of the invention for delivering medicinal fluids to a patient is there shown and generally designated by the numeral 70. This latest form of the invention is similar in many respects to that shown in FIGS. 6 through 11 and like numerals are used to identify like components. As best seen in FIG. 14, the device here comprises an elongated, tubular-shaped housing 72 having first and second ends 72a and 72b respectively. The primary difference between this latest embodiment and the earlier described embodiments resides in the provision of novel adding means for adding compounds such as drugs to a liquid contained within the device housing. Housing 72 comprises a forward portion 72c and a rearward portion 72d. Rearward portion 72d has an interior wall 72e defining a first reservoir 74 that is in communication with a second reservoir 76 via a transfer chamber 78. Second reservoir 76 is in communication with a fluid outlet 80 formed in forward portion 72c (FIG. 14). Rearward portion 72d also has an interior wall 82 that defines a third reservoir 84. A biocompatible rear closure member 86 closes the rear end portion 72b of housing 72 in the manner shown in FIG. 14 forming a hermetic seal. Closure member 86 comprises a part of the fill means of the invention for filling fluid reservoir 74.

First reservoir 74 functions to contain the medicinal fluid “F” that is to be intermixed with an additive and then delivered to the patient. Reservoir 84, on the other hand, contains a heat expandable mass 88 that is of identical character to expandable mass 24 as previously described and is readily expandable by the heat of the patient's body.

Disposed between reservoir 74 and reservoir 88 is a sealably slidable pusher means for urging the medicinal fluid contained within chamber 74 toward chamber 76 and then toward outlet 80 upon expansion of expandable mass 88. As in the earlier described embodiments, the pusher means comprises an elastomeric pusher member 90 that is disposed in sealing engagement with the internal wall 72e of housing 72.

As best seen in FIG. 14, fluid outlet 80 here comprises a small diameter generally cylindrically shaped orifice that communicates with the infusion means of the device, which is identical to that described in connection with the embodiment shown in FIGS. 6 through 11.

Located between rear closure member 86 and expandable mass 88 is an elastomeric stopper member 92 that is disposed in sealable engagement with the internal wall 82 of housing 72. Prior to installing pusher member 90, expandable mass 88 and stopper member 92, reservoir 74 can be conveniently filled with the parenteral fluid to be delivered to the patient.

The apparatus of this latest form of the invention is unique in that it provides the opportunity to add to the parenteral fluid “F” contained within the reservoir 74 selected elements, chemical compounds and biologically active materials such as drugs, medicaments, biological agents, or other therapeutic agents (additives). This addition is accomplished by removably affixing the selected additives to a support structure which can be placed within reservoir 76 and within the path of the fluid flowing through the device so that upon contact with the fluid, the additives are released at a controlled rate to the fluid.

With regard to the important adding means aspect of the invention as discussed in the preceding paragraph, the terms used therein and hereinafter have the following meanings:

Element—any of the fundamental substances that consist of atoms of only one kind and that singly or in combination constitute all matter.

Additive—the element, compound, substance, agent, biologically active material, or other material, which is to be added, all or in part, to the parenteral eluting fluid contained within reservoir 74.

Parenteral Fluid—any solution which may be delivered to a patient, including water, saline solutions, alkalizing solutions, dextrose solutions acidifying solutions, electrolyte solutions, reagents, solvents and like aqueous solutions.

Beneficial Agents—any drug, medicament, pharmaceutical, medical polymer, enzyme, hormone, antibody, element, chemical compound or other material useful in the diagnosis, cure, mitigation, treatment or prevention of disease and for the maintenance of the good health of the patient.

Biologically Active Material—a substance which is biochemically, Immunochemically, physiologically, or pharmaceutically active or reactive. Biologically active material includes at least one or more of the following: biochemical compounds (such as amino acids, carbohydrates, lipids, nucleic acids, proteins, and other biochemicals and substances which may complex or interact with biochemical compounds), such biochemical compounds biologically functioning as antibodies, antigenic substances, enzymes, cofactors, inhibitors, lectins, hormones, hormone producing cells, receptors, coagulation factors, growth enhancers, histones, peptides, vitamins, drugs, cell surface markers and toxins, among others known to those skilled in the art. Of the group of biologically active materials described, proteins are of utmost current interest because of the large molecule genetically engineered biopharmaceuticals as those species to be immobilized and congregated on the additive carriers hereinafter to be described. A discussion of the use of biomosaic polymers as carriers for biologically active materials is set forth in European Patent Application 0,430,517 A2.

Adding Means—an additive and any means for presenting the additive to the fluid flowing through the transfer chamber 78 toward reservoir 76 in a manner such that all or any part of the additive will be added to the fluid. The adding means comprises the additive and the additive presentation means which may take the form of a functional support, or carrier, an anchorage, a deposition or reaction site or an element holder with or without some type of intermediate matrix or other release composition.

Additive Presentation Means—Any means such as a functional support, scaffold or substrate for presenting the additive to the fluid flowing toward outlet 80 of the device. The functional substrate can comprise a polymer, copolymer, an inter-polymer, a ceramic, a crystal sponge, a carbon based matrix, a cellulosic, glass, plastic, silicone biomosaic polymers, azlactonefunctional polymer beads, adduct beads, carboxylate-functional polymer beads, gums, gells, filaments and like carriers.

By way of illustration, the adding means of the present form of the invention can take several different forms. However, in its preferred form, the adding means here includes a cylindrically shaped, functional support structure or scaffold which is housed within reservoir chamber 76 and to which various additives, including beneficial agents such as drugs, biologically active materials, and chemical elements and compounds can be releasably connected. These additives are carried by supporting structure 94 in a manner such that, as the liquid flows through transfer chamber 78 and circulates in, around and through the support structure, the additives will be presented to the liquid flow and efficiently added to the liquid as it flows toward outlet 80 and toward the infusion means.

The additives themselves can also take various physical forms including liquid, solid, granular, powder, particle, gel, wax, hydrocolloid carrier, gum, film, tablet, crystalline, emulsions, microcrystalline, microspherical, spray dried compounds and lyophilized compounds and saturants. The additives can be removably connected to, immobilized on, impregnated within or supported by the support means in a number of ways. The additives can be chemically or mechanically attached, affixed, or bound directly or indirectly, linked or cross linked, anchored to the surfaces of the support, or surface active agent or they can be absorbed, reaction catalyzed, electrostatically encapsulated, attached by chemical modification or transformation to the carrier surface, polymerized on or through the carrier, with or without the use of an interpolymer, localized, entrapped, suspended, deposited, impregnated, coated, or occluded or otherwise removably affixed within voids, cells, tubules, and intersticies formed in support structure 94. One important method for removably affixing the additive to the support structure includes treating the structure with a compound having selected reactive functional groups such as azlactone functional compounds with their unique ability to react with aqueous media and their high binding capacity. In this way complexing agents, catalysts and biological materials such enzymes or other proteins, as well as biomacromolecules can be attached to the carrier for later removal and recovery. Additionally, the use of one or more monomeric or polymerized surface active agents allows for rapid dissolution and smooth liberation of the additives.

Similarly, the additives can be added to or intermixed with the liquid flowing toward outlet 80 by one or more of various mechanisms, including mechanical release, chemical reaction, dissolution, disorbsion, debinding, delinking, bioseparation, diffusion, washing, disintegration, erosion, disassociation, solubilization, leeching, enzymatic cleavage, biological reaction, osmosis, separation from ring opening materials by a ring opening reaction, and other separation means. Also forming an important aspect of the delivery device of this latest form of the invention is flow control means for controlling the rate of fluid flow of medicinal fluid from chamber 76 through fluid outlet 80. This flow control means is substantially identical to that previously described herein and comprises a porous rate control frit 95 that includes a plurality of circumferentially spaced stand-off elements 95a (FIGS. 14 and 16). It should be understood that the rate of fluid flow is also controlled by the swelling kinetics of the expandable mass 84. Accordingly, the rate control frit 95 may act as a secondary rate control mechanism to fine tune the delivery rate. In certain instances, rate control frit 95 may not be required.

In using the fluid delivery device of the invention, the device is implanted in the patient's body in the manner shown in FIG. 11 with the porous tip 66 strategically positioned at the location at which the medicinal fluid is to be delivered to the patient. Once implanted, the heat of the patient's body will cause expandable mass 84 to controllably expand causing plunger 90 to be urged forwardly of reservoir 74. As the plunger moves forwardly, the fluid “F” will impinge on a third plunger 96 urging it forwardly toward transfer chamber 78. It should be noted that, since plunger 96 sealably engages the interior wall 97 of housing 72 (FIG. 14), fluid cannot pass by the plunger until the plunger enters transfer chamber 78. However, upon entering chamber 78, fluid can by pass plunger 96 and flow through a plurality of circumferentially spaced fluid passageways 78a formed in the wall of chamber 78 (FIG. 15). Provided between passageways 78a are circumferentially spaced protuberances 79, which function to center stopper 96. The fluid flowing through passageways 78a will impinge on a porous impedance frit 100 that is located between chamber 78 and chamber 76, which houses the adding means. As expandable mass 84 continues to expand, the fluid “F” will flow through impedance frit 100 and into chamber 76 which houses functional support structure 94 then flow through and about structure 94 in a manner such that the additives 102 that are removably affixed to the structure will be intermixed with the fluid. The mixture thus formed will flow outwardly of outlet 80 into the delivery tube 64 and outwardly of porous tip 66 at a precisely controlled low rate of flow where it will dose the site of therapy selected by the treating physician.

Referring to FIGS. 17 through 25, yet another form of the implantable fluid delivery device of the invention for delivering medicinal fluids to a patient is there shown and generally designated by the numeral 108. This latest form of the invention is similar in many respects to that shown in FIGS. 12 through 16 and like numerals are used to identify like components. As best seen in FIGS. 18 and 20, the device here comprises an elongated, tubular-shaped housing 110 having first and second ends 110a and 110b respectively. The primary difference between this latest embodiment and the embodiment shown in FIGS. 12 through 16 resides in the provision of several different types of additive presentation means or scaffolds for adding compounds such as drugs to a liquid contained within the device housing. Housing 110 comprises a forward portion 110c and an interconnected rearward portion 110d. Rearward portion 110d has an interior wall 110e defining a first reservoir 112. Forward portion 110c also has an interior wall 110f that defines a second reservoir 114 that is in communication with a second reservoir fluid outlet 114a via a transfer chamber 115. Rearward portion 110d also has an interior wall 110g that defines a third reservoir 118. A biocompatible rear closure member 120 closes the rear end portion 110b of housing 110 in the manner shown in FIG. 20 forming a hermetic seal.

First reservoir 112 functions to contain the medicinal fluid “F” that is to be intermixed with an additive and then delivered to the patient. Reservoir 118, on the other hand, contains a heat expandable mass 122 that is of identical character to expandable mass 24 as previously described and is readily expandable by the heat of the patient's body.

Disposed between reservoir 112 and reservoir 118 is a sealably slidable pusher means for urging the medicinal fluid contained within chamber 112 toward third chamber 114 and then toward outlet 114a upon expansion of expandable mass 122. As in the earlier described embodiments, the pusher means comprises an elastomeric pusher member 90 that is disposed in sealing engagement with the internal wall 110e of housing 110.

As best seen in FIG. 20, fluid outlet 114a here comprises a small diameter generally cylindrically shaped orifice formed in forward closure cap 124. Outlet or orifice communicates with the infusion means of the device, which is identical to that described in connection with the embodiment shown in FIGS. 6 through 11. Closure cap 124 closes the forward end of the housing and is provided with suture links 124a for tying the device in place during the implantation step.

Located between rear closure member 120 and expandable mass 122 is an elastomeric stopper member 92 of the character previously described that is disposed in sealable engagement with the internal wall 110g of housing 110.

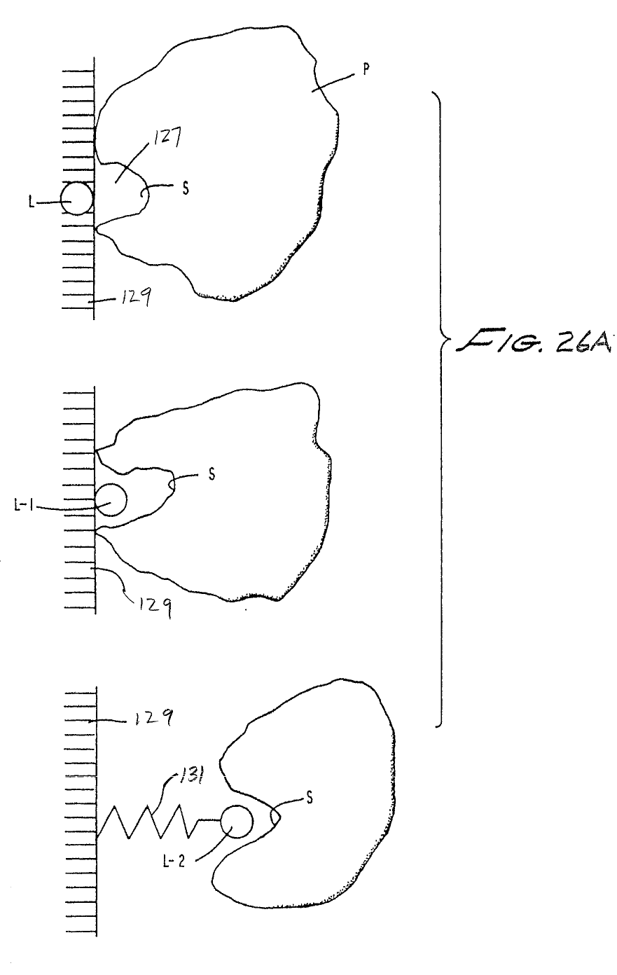

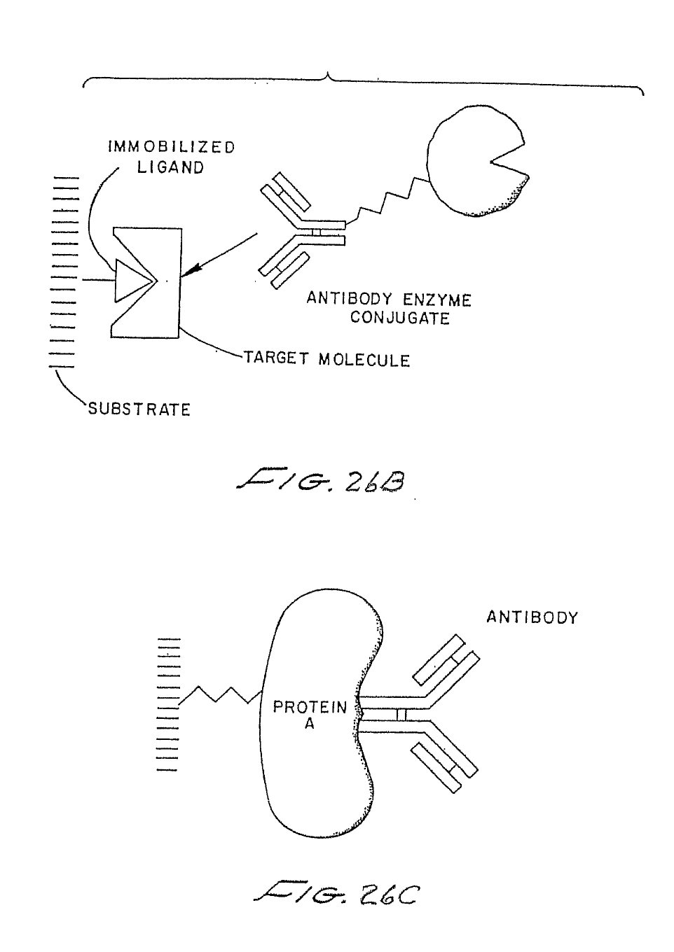

The apparatus of this latest form of the invention is unique in that it provides the opportunity to add to the parenteral fluid “F” contained within the reservoir 112 various additives of the character previously described herein as, for example, drugs, medicaments, biological agents, or other therapeutic agents. This addition is here accomplished by removably affixing the chosen additives to a selected one of several different structures or scaffold which can be placed within reservoir 114 and within the path of the fluid flowing through the device so that upon contact with the fluid, the additives are released at a controlled rate to the fluid. The novel additive presentation means or scaffolds are illustrated in FIG. 25A through 25M. For example, scaffold 25A comprises a coated wafer structure, while scaffold 25B comprises a porous ceramic. Similarly, scaffold 25C comprises an alternate sized, plugged pore structure and scaffold 25D comprises a plurality of spaced apart porous wafers. On the other hand, scaffold 25E comprises a permeable micro porous polymer structure, while scaffold 25F comprises a multiplicity of fused microspheres. Scaffold 25G comprises high porosity celulosics, scaffold 25H comprises a solid coated tube and scaffold 25I comprises a porous bundle. Similarly, scaffold 25J comprises separate coated layers laminated over a porous structure and scaffold 25K comprises a multiplicity of glass bundles forming a tortuous flow path. Finally, scaffold 25L comprises a polymer foam and scaffold 25M comprises a synthetic polymer wool.

As previously mentioned, the additives themselves can also take various physical forms including liquid, solid, granular, powder, particle, gel, wax, hydrocolloid carrier, a gum, film, tablet, crystalline, emulsions, microcrystalline, microspherical, spray dried compounds and lypohilized compounds and saturants. The additives can be removably connected to, immobilized on, impregnated within or supported by the support means in a number of ways. The additives can be chemically or mechanically attached, affixed, or bound directly or indirectly, linked or cross linked, anchored to the surfaces of the support, or surface active agent or they can be absorbed, reaction catalyzed, electrostatically encapsulated, attached by chemical modification or transformation to the carrier surface, polymerized on or through the carrier, with or without the use of an interpolymer, localized, entrapped, suspended, deposited, impregnated, coated, or occluded or otherwise removably affixed within voids, cells, tubules, and intersticies formed in the support. One important method for removably affixing the additive to the functional support means includes treating the functional support means with a compound having selected reactive functional groups such as azlactone functional compounds with their unique ability to react with aqueous media and their high binding capacity. In this way complexing agents, catalysts and biological materials such enzymes or other proteins, as well as biomacromolecules can be attached to the carrier for later removal and recovering. Additionally, the use of one or more monomeric or polymerized surface active agents allows for rapid dissolution and smooth liberation of the additives. A discussion of such surface-active agents is contained in U.S. Pat. No. 4,963,367 issued to Ecano. Reference should also be made to U.S. Pat. No. 5,279,558 issued to the present inventor. Because of the pertinence of patent No. 5,279,558, this patent is hereby incorporated by reference as though fully set forth herein.

Similarly, the additives can be added to or intermixed with the liquid flowing through the device by one or more of various mechanisms, including mechanical release, chemical reaction, dissolution, disorbsion, debinding, delinking, bioseparation, diffusion, washing, disintegration, erosion, disassociation, solubilization, leeching, enzymatic cleavage, biological reaction, osmosis, separation from ring opening materials by a ring opening reaction, and other separation means.

Additionally, a polymer can be used as the carrier or support for some component of a reaction system. Three classes of polymeric supports can be used, namely polymeric reagents, polymeric catalysts and polymeric substrates. A discussion of polymers as carriers or supports is contained in Principles of Polymerization, Second Edition by George Odian. Microporous polymers usable as carriers are also fully described in U.S. Pat. No. 4,519,909 issued to Castro.

With respect to each of the foregoing embodiments of the invention, materials suitable for the construction of tubular members 16, 42 and 72 can comprise glass, plastic or metals such as stainless steel and titanium. The various elastomeric pusher members can be constructed from various polymers, including silicone material and synthetic latex, butyl rubber and polyisoprene.

In addition, the additives can be removably affixed to the functional support means in various ways to enable the use of separation techniques broadly defined by the term chromotography. Chromotography as used herein refers to a group of separation techniques which are characterized by a distribution of the molecules to be separated between two phases, one stationary and the other mobile. Affinity chromatography involves the use of biological interactions and contemplates the use of affinity chromatography supports through which the eluting fluid flows. In the present embodiment of the invention, the additive presentation means assumes the character of an affinity chromatography support to which various ligands are attached. In the practice of affinity chromotography techniques, one of the members of the pair in the interaction, the ligand, is immobilized on a solid phase, while the other, the counterligand (most often a protein), is absorbed from the extract that is passing the substrate during the manufacturing process. Importantly, affinity chromatography techniques can include the use of highly versatile azlactone functional compounds, such as azlactone functional beads, as well as the use of a wide variety of other media for activation and coupling chemistry. Examples of ligands that can be attached to the affinity supports include antibodies, enzymes, lectins, nucleic acids hormones and vitamins. Examples of important counterligands include antigens, virus, cells, cell surface receptors and the like. Chromotography and affinity chromatography techniques are described in detail in Protein Purification by Janson and Ryden, Copyright 1989 and reference should be made to this work to provide a working understanding of the techniques.

Polymeric azlactones are well known in the prior art. Their use in the production of homopolymers and copolymers has been described in a number of patents. See for example, U.S. Pat. No. 3,488,327 (issued Jan. 6, 1970 to F. Kollinsky et al.); U.S. Pat. No. 3,583,950 (issued Jun. 8, 1971 to F. Kollinsky et al.); and U.S. Pat. No. 4,304,705 (issued Dec. 8, 1981 to S. M. Heilmann et al.). Azlactones, or oxazolones, are cyclic anhydrides of N-acylamino acids and have been used extensively in organic synthesis. The formation of a five-membered azlactone of particularly useful functionality for immobilization purposes can be accomplished through the reaction of a carboxylate group with a-methyl alanine using a two-step process. (See Immobilized Affinity Ligand Techniques-Hermanson, Mallia and Smith, Copyright 1992). One method of forming azlatone beads, the use of which has been previously mentioned herein, makes use of this process in the polymerization of monomers to first yield a carboxyl group on the matrix. In the second step, the azlactone ring is formed in anhydrous conditions through the use of a cyclization catalyst. Suitable cyclization agents that will drive this reaction include acetic anhydride, alkyl chloroformates, and carbondiimides. The process of forming these active groups and of making beaded polymeric supports containing them has been thoroughly described in patents assigned to 3M Corporation (U.S. Pat. Nos. 4,871,824 and 4,737,560). These support materials are now available under the tradename “Emphase”. U.S. Pat. Nos. 5,045,615 and 5,013,795 which have been assigned to 3M Corporation also describe recent advances in this technology.

As pointed out in the 3M Corporation U.S. Pat. No. 4,737,560, azlactone-functional polymer beads are useful reactive supports for the attachment of functional materials to provide novel adduct beads. The adduct beads are useful as complexing agents, catalysts, reagents, and as enzyme or other protein-bearing supports. The term “support” or “affinity support” as used in this sense is usually understood to refer to a combination of (1) a ligand (usually of some known molecular configuration), that is firmly attached (e.g., immobilized), often by covalent means, and (2) a matrix (usually a solid insoluble substance). Azlactone support matrix materials and coupling chemistry is also of special interest because of its accessible matrix surface area and effective ligand diversity that can be attached to that surface.

U.S. Pat. No. 4,072,566 issued to Lynn on Feb. 7, 1978, and entitled “Immobilized Biologically Active Proteins” discloses a method of bonding enzymes or other biologically active proteins to an inorganic support material using pphenylenediamine. The support materials disclosed as useful in the invention include siliceous materials, stannic oxide, titania, manganese dioxide, and zirconia. The functional support structures of the present invention can take on the character of affinity supports and are uniquely constructed to permit enzymes or other biologically active proteins to be bound thereto for later removal. This is accomplished by treating the functional supports in the manner disclosed in the prior art patents identified in the preceding paragraphs with a compound having selected reactive functional groups such as azlactone functional compounds. In this way complexing agents, catalysts and biological materials such enzymes, proteins or other affinity absorbants, as well as biomacromolecules can be attached to the carrier for later removal and recovering.

When attaching certain biologically active proteins and other macro molecules, the use of spacer arms or leashes have been found to be very beneficial. Spacer arms or leashes are low-molecular-weight molecules that are used as intermediary linkers between a support material and an affinity ligand. Usually spacers consist of linear hydrocarbon chains with functionalities on both ends for each coupling to the support and ligand. First, one end of the spacer is attached chemically to the matrix using traditional immobilization chemistries; the other end is connected subsequently to the ligand using a secondary coupling procedure. The result is an immobilized ligand that sticks out from the matrix backbone by a distance equal to the length of the spacer arm chosen.

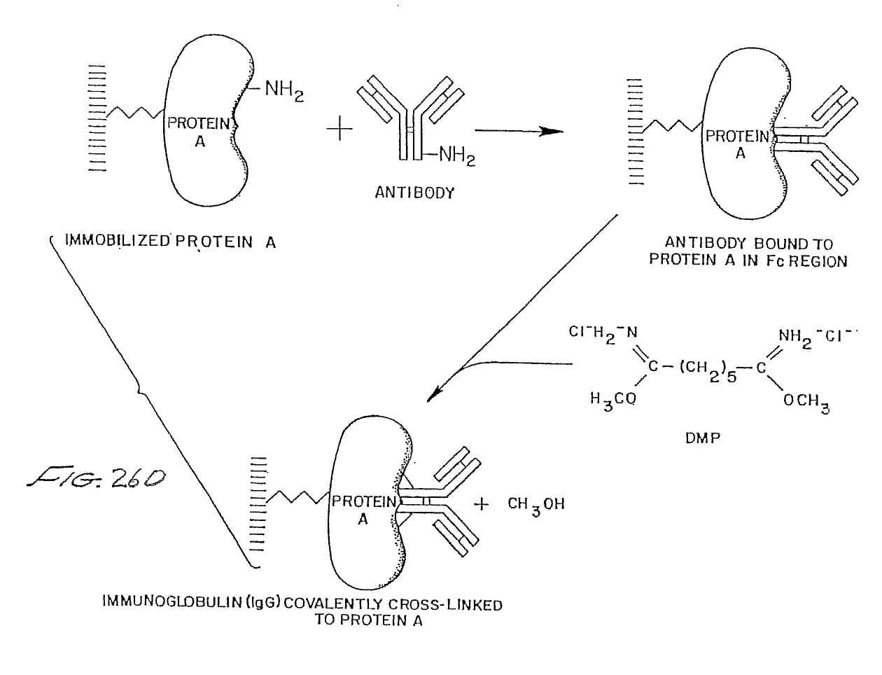

Referring to FIGS. 26A, 26B, 26C, and 26D, the use of spacer arms to attach proteins and enzymes to the substrate is there schematically illustrated. The principal advantage of using a spacer arm is that it provides ligand accessibility to the binding site of a target molecule. When the target molecule is a protein with a binding site somewhat beneath its outer surface, a spacer is essential to extend the ligand out far enough from the matrix to allow interaction. As indicated in FIG. 26A, when the ligand binding site S is buried or in a pocket 127 located just below the surface of the protein P, a ligand L that is either below the surface of the support material 129 (upper portion) or a ligand L-1 that is attached directly to the surface (middle portion) cannot reach the level of the binding site S on an approaching protein molecule. The result may be weakened interaction or no binding at all. Accordingly, in these instances, spacer arm 131 is required to provide the ligand L2 accessibility to the binding site of the protein molecule (lower portion of FIG. 26A). The details covering the use of spacer arms are fully set forth in Section 3.1.1 of the previously referred to work entitled Immobilized Affinity Ligand Techniques. This Section 3.1.1 is incorporated herein by reference.

Turning now to FIGS. 26B, 26C, and 26D, it is to be noted that immobilized protein A can be used to immobilize an antibody molecule by taking advantage of the natural affinity of protein A for immunoglobulins. Incubation of a specific antibody with protein A matrix will bind the antibody in the Fe region, away from the antigen binding sites. Subsequent cross-linking of this complex with DMP (dimethyl pimelimidate) yields a covalently attached antibody with the antigen binding sites facing outward and free to interact with antigen.

With rigid support materials, a spacer molecule may also provide greater flexibility, allowing the immobilized ligand to move into position to establish the correct binding orientation with a protein. The degrees of freedom that a hydrocarbon extender can provide are much greater than the movement possible within the polymeric backbone of a matrix.

The choice of spacer molecule can affect the relative hydrophilicity of the immediate environment of an immobilized ligand. Molecules containing long hydrocarbon chains may increase the potential for nonspecific hydrophobic interactions, especially when the affinity ligand is small and of low molecular weight. Selecting spacers that have more polar constituents, such as secondary amines, amide linkages, ether groups or hydroxyls will help keep hydrophobic effects at a minimum.

It is also important to consider the ionic effects a spacer molecule may impart to a gel. Spacers with terminal primary amine groups should be completely coupled with ligand or blocked by a nonrelevant molecule (e.g., acetic anhydride; see Section 3.1.1.9 of Immobilized Affinity Ligand Techniques) to eliminate the potential for creating a positive charge on the support. With small ligands, these residual charges can form a secondary environment that may cause considerable nonspecific interactions with proteins. The same holds true for spacers with terminal carboxylic groups. In general, a negatively charged spacer will cause less nonspecific protein binding than a positively charged one, but blocking excess remaining groups is still a good idea. A good blocking agent for use with carboxylic residues is ethanolamine, which leaves a terminal hydroxyl group (See Immobilized Affinity Ligand Techniques for an expanded discussion of types of spacers and various immobilization and coupling protocols.) As pointed out in Protein Purification, Janson and Ryden, Copyright 1989 which describes some alternate forms of protein immobilization at Page 310: “Ligand-protein interaction is often based on a combination of electrostatic, hydrophobic and hydrogen bonds. Agents which weaken such interactions might be expected to function as effective non-specific eluants.”This work provides further teaching of the techniques described herein. It is important to recognize that, as used in the present form of the invention, affinity supports are now capable of total binding capacity at a level that enables attachment to the support of additives in substantial amounts for subsequent release, recovery and infusion of beneficial agents in a manner which can be therapeutically efficatious to a patient.

In using the fluid delivery device of this latest form of the invention, the device is implanted in the patient's body in the manner shown in FIG. 11 with the porous tip strategically positioned at the location at which the medicinal fluid is to be delivered to the patient. Once implanted, the heat of the patient's body will cause expandable mass 118 to controllably expand causing plunger 90 to be urged forwardly of the reservoir 124. As the plunger moves forwardly, the fluid “F” will impinge on a third plunger 125a urging it forwardly toward a transfer chamber 116. As in the earlier described embodiments, since plunger 125a sealably engages the interior wall of housing 110d (FIG. 20), fluid cannot pass by the plunger until the plunger enters transfer chamber 116. However, upon entering chamber 116, fluid can by pass plunger 125a and flow through fluid passageways 110p formed in the wall of chamber 116 (FIG. 20). The fluid flowing through these passageways will impinge on a porous impedance frit 100 that is located between chamber 116 and the chamber that houses the adding means. As expandable mass 118 continues to expand, the fluid “F” will flow through impedance frit 100 and will then flow through and about the selected scaffold that is disposed within chamber or reservoir 114. As the fluid flows around, about and through the scaffold, the additives that are removably affixed to the scaffold will be intermixed with the fluid. The mixture thus formed will flow through frit 95 where it will impinge on a second sealing plunger 125b urging it into transfer chamber 115. When plunger 125b is positioned within the transfer chamber the fluid mixture can by pass the plunger and flow outwardly of outlet 114a and into infusion line 64.

Having now described the invention in detail in accordance with the requirements of the patent statues, those skilled in this art will have no difficulty in making changes and modifications in the individual parts or their relative assembly in order to meet specific requirements or conditions. Such changes and modifications may be made without departing from the scope and spirit of the invention, as set forth in the following claims.

I claim:

1. An implantable delivery device for delivering a beneficial agent to selected site comprising an elongated housing having an outlet and an interior wall defining first and second reservoirs, said first reservoir containing the beneficial agent and being in communication with the fluid outlet, said device comprising:
(a) a heat expandable mass contained within said second reservoir, said heat expandable mass being expandable by the heat of the patient's body; and
(b) means disposed within the housing intermediate the first and second reservoirs for urging the beneficial agent toward the outlet upon expansion of said heat expandable mass.

2. The device as defined in claim 1 further including flow control means disposed intermediate the first reservoir and the selected site for controlling fluid flow toward the selected site.

3. The device as defined in claim 1 further including infusion means connected to said housing and communicating with the fluid outlet for infusing said medicinal fluid into the patient.

4. The device as defined in claim 1 in which said heat expandable mass comprises a polymer.

5. The device as defined in claim 1 in which said heat expandable mass comprises a gel.

6. The device as defined in claim 1 in which said heat expandable mass comprises an ionized acrylamide gel composition.

7. The device as defined in claim 1 in which said heat expandable mass comprises an interpenetrating polymer network.

8. The device as defined in claim 1 further including flow control means for controlling flow of the beneficial agent toward the selected site, said flow control means comprising said expandable mass.

9. The device as defined in claim 1 further including adding means housed within the housing for adding an additive to the fluid contained within the first reservoir.

10. The device as defined in claim 1 in which said means for urging the beneficial agent toward the selected site comprises a pusher member disposed in sealable engagement with the interior wall of the housing.

11. The device as defined in claim 1 in which said housing comprises an elongated tubular member having first and second open ends.

12. The device as defined in claim 11, further including a closure cap closing said second open end of said elongated tubular member.

13. The device as defined in claim 11, further including cover means covering said tubular member, said cover means including a cover having an end wall extending over said first open end of said tubular member.

14. The device as defined in claim 12 in which said infusion means comprises an elongated delivery line having first and second ends, said first end being connected to said end wall of said cover.

15. The device as defined in claim 14 further including a porous delivery tip connected to said second end of said delivery line.

16. An implantable fluid delivery device for delivering medicinal fluids to a patient comprising an elongated housing having first and second ends, a fluid outlet formed in said first end and an interior wall defining first and second reservoirs, said first reservoir containing fluid and being in communication with the fluid outlet, said device comprising:
(a) an expandable gel contained within said second reservoir;
(b) means disposed within the housing intermediate the first and second reservoirs for urging said medicinal fluid toward the fluid outlet upon expansion of said heat expandable gel; and
(c) adding means disposed within said housing between said first reservoir and said fluid outlet for adding an additive to the fluid contained within said first reservoir.

17. The device as defined in claim 16 further including flow control means comprising a porous frit disposed intermediate the fluid outlet and the first reservoir for controlling fluid flow through the fluid outlet.

18. The device as defined in claim 16, in which said adding means comprises a functional support structure housed within said housing and in which said additive comprises a drug removably affixed to the functional support structure.

19. The device as defined in claim 16 in which said additive is substantially removable from said support using affinity chromatography techniques.

20. The device as defined in claim 19 in which a ligand is connected to said support and a target molecule is connected to said ligand.

21. A device as defined in claim 20 in which a spacer arm is connected to said support and in which a ligand is connected to said spacer arm.

22. The device as defined in claim 21 in which an enzyme is connected to said target molecule.

23. The device as defined in claim 22 in which said target molecule is a protein.

24. The device as defined in claim 23 in which an antibody is bound to said protein.

25. The device as defined in claim 16, further including infusion means connected to said housing and communicating with the fluid outlet for infusing said medicinal fluid into the patient.

26. The device as defined in claim 16 in which said housing further includes an elongated tubular member having first and second open ends.

27. The device as defined in claim 26, further including cover means covering said tubular member, said cover means including a cover having an end wall extending over said first open end of said tubular member.

28. The device as defined in claim 26 in which said infusion means comprises an elongated delivery line having first and second ends, said first end being connected to said end wall of said cover.

29. The device as defined in claim 23, further including a porous delivery tip connected to said second end of said delivery line.

30. An implantable drug delivery device for delivering medicinal fluids to a patient comprising a housing having a fluid outlet and including an interior wall defining first, second and third reservoirs, said first reservoir being in communication with the fluid outlet and containing a fluid, said device comprising:
(a) a heat expandable polymer contained within said third reservoir, said heat expandable polymer being expandable by the heat of the patient's body;
(b) adding means contained within said second reservoir for adding an additive to the fluid contained within the first reservoir;
(c) means disposed within the housing intermediate the first and third reservoirs for urging said medicinal fluid toward the second reservoir upon expansion of said heat expandable member; and
(d) infusion means connected to said end wall of said cover and communicating with said fluid outlet for infusing said medicinal fluid into the patient.

31. The device as defined in claim 30 further including flow control means comprising a porous frit disposed intermediate the fluid outlet and the first reservoir for controlling fluid flow through the fluid outlet.

32. The device as defined in claim 30 in which said heat expandable polymer comprises a gel.

33. The device as defined in claim 30 in which said heat expandable polymer comprises a phase-transition gel.

34. The device as defined in claim 30 in which said heat expandable polymer exhibits swelling kinetics that can be tailored to control the rate of delivery of the medicinal fluids to the patient.

35. The device as defined in claim 30 in which said additive comprises a beneficial agent.

36. The device as defined in claim 30 in which said additive comprises a drug.

37. The device as defined in claim 30 in which said housing further includes a transfer chamber disposed between said first reservoir and said third reservoir.

38. The device as defined in claim 30 in which said infusion means comprises an elongated delivery line having first and second ends, said first end being connected to said fluid outle of said housing.

39. The device as defined in claim 38, further including a porous delivery tip connected to said second end of said delivery line.

40. The device as defined in claim 38, further including a closure cap closing said second end of said tubular member.

41. The device as defined in claim 38, further including a stopper member disposed intermediate said closure cap and said third reservoir.
